(19) United States
(12) Patent Application Publication
(10) Pub. No.: US 2026/0294762 A1
(43) Pub. Date: Oct. 1, 2026

(54) SKIN CARE COMPOSITION

(71) Applicant: DSM IP Assets B.V., Maastricht (NL)

(72) Inventors: Anne JANSSEN, Kaiseraugst (CH); Karina RADOMSKY, Kaiseraugst (CH)

(21) Appl. No.: 19/164,375

(22) PCT Filed: Mar. 12, 2024
(86) PCT No.: PCT/EP2024/056533

§ 371 Date: Sep. 11, 2025

(30) Foreign Application Priority Data
Mar. 16, 2023 (EP) 23162363.8

(51) Int. Cl. A61K 8/37; A61K 8/06; A61K 8/34; A61Q 17/04; A61Q 19/00

(52) U.S. Cl. CPC A61K 8/37; A61K 8/062; A61K 8/345; A61Q 17/04; A61Q 19/00

(57) ABSTRACT

The present invention relates to cosmetic compositions containing an alkandiol and one or more cosmetic oils, characterized in that at least one of the cosmetic oils is isoamyl caprylate caprate. Said compositions exhibit an improved water repellency (lotus effect) and sensory attributes.

The present invention relates to cosmetic compositions containing an alkandiol and one or more cosmetic oils, characterized in that at least one of the cosmetic oils is isoamyl caprylate caprate. Said compositions exhibit an improved water repellency (lotus effect).

The leaf of the lotus plant exhibits remarkable water-repellency and self-cleaning properties. Although lotus plants prefer to grow in muddy rivers and lakes, the leaves and flowers remain clean and are essentially non-wettable. The lotus plant achieves this effect by producing leaves and flowers with extremely hydrophobic surfaces. When the leaves come in contact with water, the water droplets contract into substantially spherical beads which roll off the surface, sweeping away any particles of dirt they encounter.

The conventional approach to formulating water-proof or water-resistant cosmetic compositions relies on the use of hydrophobic film film forming polymers. These film forming polymers, however, often render the product unattractive for the end consumer due to the resulting sensory properties as such products often exhibit an oily, dull, and sticky feeling on the skin as e.g. outlined in DE 102010063825.

Thus, there is an ongoing need for aesthetically pleasing cosmetic compositions exhibiting an improved water repellancy e.g. to enhance water resistance of cosmetic composition.

Surprisingly, it has now been found, that the combined use of isoamyl caprylate caprate and an alkandiol significantly improves the water repellency (lotus effect) of a composition while even improving the sensory properties thereof.

Thus, in a first embodiment, the present invention provides cosmetic compositions comprising an alkandiol and one or more cosmetic oils, wherein at least one of the cosmetic oils is isoamyl caprylate caprate.

Another subject matter of the invention is directed to the use of an alkandiol for increasing the water repellency of a cosmetic composition comprising one or more cosmetic oils, wherein at least one of the cosmetic oils is isoamyl caprylate caprate.

In a further aspect, the invention relates to a method of improving the water repellency of a cosmetic compositions, said method encompassing the step of preparing a cosmetic composition comprising both of an alkandiol and isoamyl caprylate caprate, applying said composition to the skin, allowing the volatile constituents to evaporate, thereby forming a hydrophobic film and optionally appreciating the effect.

Isoamyl caprylate caprate is also known as fatty acids, C8-10 (even numbered), 3-methylbutyl ester (EC number: 694-886-1; CAS number: 1365095-43-7) and is available under the tradename Oxysmooth CP. It is derived from sugarcane sugar cane and mainly C8 and C10 vegetable fatty acids in a ratio of approximately 1:1 (such as in particular in the range of 1:0.9 to 0.9:1). Specifically, the term isoamyl caprylate caprate refers to a mixture consisting essentially of of isoamyl caprylate (C8) (in amounts from about 45-55%) and isoamyl caprylate (C10) (in amounts from about 40-50%). It is well understood, that the term ‘consisting essentially of’ as used herein means that the total amount of the ingredients ideally sums up to 100 wt.-percent. It is however not excluded that small amounts of impurities, such as isoamyl caproate and/or isoamyl laurate (C12) derived from the production process may be present up to a maximum of at most 10%, preferably up to 7.5%, i.e. in the range of 0-10, respectively 0-7.5% as measured by GC-MS area-% analysis.

In all embodiments of the present invention, the (total) amount of isoamyl caprylate caprate in the cosmetic compositions according to the present invention is advantageously at least 2.5 wt. %, preferably at least 5 wt.-%, most preferably at least 7.5 wt.-%, such as at least 10 wt.-% based on the total weight of the composition.

In all embodiments of the present inventon, the (total) amount of isoamyl caprylate caprate in the cosmetic compositions according to the present invention is less or equal to 30 wt.-%, preferably less or equal to 25 wt.-%, even more preferably less or equal to 20, such as less or equal to 15 wt.-%, based on the total weight of the composition.

Even more preferably, in all embodiments of the present invention, the amount of isoamyl caprylate caprate in the cosmetic compositions according to the present invention is selected in the range from 5 to 30 wt.-%, more preferably from 7.5 to 20 wt.-%, most preferably from 10 to 15 wt.-%. Further suitable ranges are selected in the range from 5 to 25 wt.-%, from 5 to 20 wt.-%, from 5 to 15 wt.-%, from 7.5 to 25 wt.-%, from 7.5 to 20 wt.-%, from 7.5 to 15 wt.-%, from 10 to 30 wt.-%, from 10 to 25 wt.-% or from to 10 to 20 wt.-%, based on the total weight of the composition.

Suitable alkanediols in all embodiments of the present invention encompass 1,2-alkanediols and 1,3-alkandiols such as in particular 1,2-butandiol, butylene glycol, 1,2-pentanediol, 1,2-hexanediol, 2-methyl-1,3-propanediol and 1,3-propanediol. Preferred in all embodiments of the present invention is the use of 1,3-propanediol, butyleneglycol and/or pentyleneglycol. Most preferred in all embodiments of the present the composition comprises as alkanediol 1,3-propandiol, butylene glycol or pentyleneglycol, preferably in the absence of any further alkanediol(s). Even more preferably, the compositions comprise butylene glycol or pentyleneglycol, even more preferably as sole alkanediol, as these lead to a particular beneficial improvement of the water repellancy.

The (total) amount of the alkanediol(s) in the cosmetic compositions according to the present invention is preferably selected in the range from 1 to 10 wt.-%, more preferably from 1.5 to 7.5 wt.-%, most preferably from 2 to 6 wt.-%, such as in the range of 2 to 5 wt.-%, based on the total weight of the composition. Further suitable ranges include from 2.5 to 5 wt.-%, or from 2.5 to 4 wt.-% such as from 2.5 to 3.5 wt.-%.

In a particular preferred embodiment, the cosmetic composition according to the present invention comprises from 10 to 15 wt.-% of isoamyl caprylate caprate and from 2 to 5 wt.-% of alkandiol(s), based on the total weight of the composition.

In all embodiments of the present invention, the weight-ratio of isoamyl caprylate caprate to alkandiol is preferably selected in the range from 1:1 to 25:1, preferably in the range from 2.5:1 to 20:1, most preferably in the range from 4:1 to 10:1. Further suitable ranges are from 3:1 to 10:1 and from 4:1 to 7.5:1.

As used herein the term ‘cosmetic oils’ refers to compounds suitable for cosmetic use having a melting point of less than about 30° C. and which are generally insoluble in water and include a hydrophobic moiety, such as in particular a hydrocarbon group with at least six carbons.

The hydrophobic moiety may include linear, cyclic, aromatic, saturated or unsaturated groups having a carbon chain of at least six carbons in which none of the six carbons is a carbonyl carbon or has a hydrophilic moiety (defined below) bonded directly thereto. The cosmetic oil preferably includes fatty acid or fatty alcohol moieties. Also preferably, the cosmetic oil does not include hydrophilic moieties, such as anionic, cationic or zwitterionic or non ionic groups which are polar groups including sulfate, sulphonate, carboxylate, phosphate, ammonium including mono-, di-, and trialkyl ammonium species, pyridinium, imidazolinium, amidinium, poly(ethyleneiminium), ammonioalkylsulfonate, ammonioalkylcarboxylate, amphoacetate, Particular suitable cosmetic oils next to isoamyl caprylate caprate to be used in the cosmetic compositions according to the present invention include C₁₂-C₁₅ alkyl benzoate, dicaprylate/dicaprate, phenethyl benzoate, dibutyl adipate, diisopropyl sebacates, dicaprylyl carbonate, di-C₁₂₋₁₃ alkyl tartrates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol, ethylhexyl cocoates. Preferably, the composition according to the present invention comprises at least on further oil next to isoamyl caprylate caprate, which one or more oil(s) is even more preferably selected from the group consisting of dibutyl adipate, phenethyl benzoate, dicaprylyl carbonate, C₁₂-C₁₅ alkylbenzoate, caprylyl carbonate, capric/caprylic triglyceride as well as mixtures thereof, most preferably the aditional oil used in combination with isoamyl caprylate caprate is C₁₂-C₁₅ alkylbenzoate. Even more preferably, the sunscreen compostions according to the present invention solely comprises isoamyl caprylate caprate and C₁₂-C₁₅ alkylbenzoate as cosmetic oils.

The total amount of the one or more additional oils (in addition to isoamyl caprylate caprate) is preferably selected in the range of 5 to 30 wt.-%, preferably in the range of 7.5 to 20 wt.-%, most preferably In the rang of 10 to 20 wt.-%.

It is furthermore preferred in all embodiments of the present invention, that the weight-ratio of the one or more additional oils such as in particular of C₁₂-C₁₅ alkylbenzoate to isoamyl caprate caprylate is selected in the range from 5:1 to 1:5, preferably from 2.5:1 to 1:2.5, most preferably from 1.5:1 to 1:1.5.

The total amount of cosmetic oils (including the isoamyl caprylate caprate) in the cosmetic compositions according to the present invention is preferably selected in the range from 10 to 50 wt.-%, preferably from 15 to 45 wt.-%, most preferably from 20 to 40 wt.-%, such as from 25 to 35 wt.-%, based on the total weight of the composition.

In a preferred embodiment, the compositios according to the present invention are emulsions.

The term ‘emulsion’ as used herein refers to compositions containing an oily phase and an aqueous phase such as in particular an O/W, W/O, Si/W, W/Si, O/W/O, W/O/W multiple or a pickering emulsions.

The amount of the oily phase in the emulsions according to the present invention (i.e. the phase containing all cosmetic oils and fats as well as all oil souble components of the composition) present in such emulsions is preferably at least 25% wt.-%, such as in the range from 25 to 70 wt.-%, preferably in the range from 30 to 60 wt.-%, most preferably in the range from 30 to 50 wt.-%, based on the total weight of the composition.

According to one even more preferred embodiment, the cosmetic compositions according to the present invention as outlined herein are O/W emulsions comprising an oily phase dispersed in an aqueous phase in the presence of an O/W emulsifier. The preparation of such O/W emulsions is well known to a person skilled in the art and illustrated in the examples.

In all embodiments of the present invention it is furthermore advantageous if the O/W emulsifier is a phosphate ester emulsifier. Among the preferred phosphate ester emulsifier are C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-15 Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier according to the invention is potassium cetyl phosphate e.g. commercially available as Amphisol® K at DSM Nutritional Products Ltd Kaiseraugst.

Further suitable O/W emulsifiers according to the present invention encompass PEG 30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Also suitable are polyalkylenglycolether such as Brij 72 (Polyoxyethylen(2)stearylether) or Brij 721 (Polyoxyethylene (21) Stearyl Ether e.g. available at Croda.

Particularly advantageous O/W emulsifiers according to the present invention are one or more of Polyglyceryl-3 Methylglucose Distearate, Lauryl Glucoside (and) Polyglyceryl-2 Dipolyhydroxystearate, Glyceryl Sterate Citrate, Sodium Cetearyl Sulfate, Cetearyl Glucoside; Polyglyceryl-6 Stearate (and) Polyglyceryl-6 Behenate, Cetearyl Olivate (and) Sorbitan Olivate, Arachidyl Alcohol (and) Behenyl Alcohol (and) Arachidyl Glucosides, Cetearyl Alcohol (and) Coco-Glucoside, Coco-Glucoside (and) Coconut Alcohol, PEG-100 Stearate (and) Glyceryl Stearate, Sodium Stearoyl Glutamate, Steareth-20, Steareth-21, Steareth-25, Steareth-2, Ceteareth-25 and Ceteareth-6 (all listed by their INCI names).

It is particularly preferred in accordance with the invention if the compositions comprises potassium cetyl phosphate as emulsifier.

The at least one O/W respectively Si/W emulsifier with all the definitions and preferences as given herein is preferably used in an amount of 0.5 to 10 wt. % such as in particular in the range of 0.5 to 5 wt.-% such as most in particular in the range of 0.5 to 4 wt.-% such as in the range of 1 to 4 wt.-%, 1 to 3 wt.-% or 2 to 3 wt.-%, based on the total weight of the composition.

Suitable W/O- or W/Si-emulsifiers according to the present invention are are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weigh of the composition.

The cosmetic compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-% or in the range of 2 to 4 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), polyhydroxystearic acid, glyceryl stearate, glyceryl myristate (Estol 3650), hydrogenated coco-glycerides (Lipocire Na10) as well as mixtures thereof.

In all embodiments of the present invention, it is particular advantageous if the composition also comprises cetearyl alcohol and/or stearyl alcohol, preferably both thereof, most preferably in an amount (total) of 1 to 5 wt.-%, preferably of 2 to 4 wt.-%, based on the total weight of the composition.

In a still further advantageous aspect of the invention, the cosmetic compositions of the present invention further comprise a preservative and/or a preservative booster preferably selected from the group consisting of ethanol, phenoxyethanol, 1,2-pentanediol, 1,2-hexanediol, 1,2-octanediol (caprylyl glycol), 1,2 decanediol, 2-methyl-1,3-propanediol, p-hydroxyacetophenone, more preferably selected from the group consisting of ethanol, phenoxyethanol, ethylhexylglycerin, hexylglycerin, glyceryl caprylate as well as mixtures thereof, most preferably selected from the group of phenoxyethanol and ethylhexyl glycerine as well as mixtures thereof. When present, the preservative respectively the preservative booster is preferably used in an amount of 0.01 to 2 wt. %, more preferably in an amount of 0.05 to 1.5 wt.-%, most preferably in an amount of 0.1 to 1.5 wt.-%, such as in the range of 0.5 wt.-% to 1.5 wt.-% or 0.75 to 1.25 wt.-%, based on the total weight of the composition.

In a particualar advantageous embodiment, the composition of the invention comprise phenoxyethanol and/or ethylhexylglycerin, preferably phenoxyethanol and ethylhexylglycerin.

The cosmetic composition may further comprise commonly used UVA, UVB or broadband UV-filter substances. Such compositions are commonly referred to as sunscreens.

In a particular advantageous embodiment the compositions according to the present invention further comprise Bis-Ethylhexyloxyphenol methoxyphenyl triazine (INCI, also referred to herein as BEMT) as UV-filter substance, as the addition thereof further increases the water repellancy of the composition as illustrated in the examples.

BEMT is also known as 2,4-bis{[4-(2-ethylhexyloxy)-2-hydroxy]phenyl}-6-(4-methoxyphenyl)-1,3,5-triazine (chemical name) or bemotrizinol (INN). BEMT acts as a broad-spectrum UV filter absorbing UVB as well as UVA rays. It has two absorption peaks, 310 and 340 nm.

BEMT is suggested for use in sun, day care, alphabetic products such as BB cream and whitening products.

In all embodiments of the present invention, the amount of BEMT in the cosmetic compositions according to the present invention is advantageously selected in the range from 0.4 to 10 wt.-%, preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 2 to 5 wt.-%, from 2 to 4 wt.-% or from 2 to 3 wt.-%, based on the total weight of the composition.

Preferably, the weight-ratio of isoamyl caprylate caprate to BEMT is selected in the range from 1:1 to 25:1, preferably in the range from 2.5:1 to 20:1, most preferably in the range from 4:1 to 10:1. Further suitable ranges are from 3:1 to 10:1 and from 4:1 to 7.5:1.

Preferably, in all embodiments of the present invention the weight-ratio of the alkandiol(s) to BEMT is selected in the range from 0.5:5 to 5:0.5, preferably in the range from 0.5:3 to 3:0.5, most preferably in the range from 5:1 to 1:1. Further suitable ranges are from 4:1 to 1:1 and from 3:1 to 1:1 and from 2:1 to 1:1. Preferably, in all embodments of the present invention the alkandiol(s) is used in an excess comapred to the amount of BEMT, i.e. in a weight-ratio of >1.

Preferably, in all embodiments of the present invention, the composition further comprises butylmethoxy dibenzoylmethane and/or Diethylamino hydroxybenzoyl hexyl benzoate (DHHB).

The amount of the butylmethoxy dibenzoylmethane, commercially available as PARSOL@1789, in the cosmetic compositions according to the present invention is preferably selected in the range from 1 to 10 wt.-%, more preferably from 1.5 to 7.5 wt.-%, most preferably from 2 to 6 wt.-%, such as in the range of 2 to 5 wt.-%, based on the total weight of the composition.

The amount of the diethylamino hydroxybenzoyl hexyl benzoate, commercially available as Uvinul A plus, in the cosmetic compositions according to the present invention is preferably selected in the range from 1 to 10 wt.-%, more preferably from 1.5 to 7.5 wt.-%, most preferably from 2 to 6 wt.-%, such as in the range of 2 to 5 wt.-%, based on the total weight of the composition.

In one embodiment it is preferred that the compositions comprise BEMT and butyl methoxydibenzoylmethane, preferably in the absence of DHHB, even more preferably as sole UV-filters.

In another embodiment it is preferred that the compositions comprise BEMT and diethylamino hydroxybenzoyl hexyl benzoate, preferably in the absence of butyl methoxydibenzoylmethane, even more preferably as sole UV-filters.

As the cosmetic compositions according to the invention are intended for topical application, it is well understood that they comprise a physiologically acceptable medium, i.e. a medium compatible with keratinous substances, such as the skin, mucous membranes, and keratinous fibres. In particular, the physiologically acceptable medium is a cosmetically acceptable carrier.

The term ‘cosmetically acceptable carrier’ as used herein refers to all carriers and/or excipients and/or diluents conventionally used in topical cosmetic compositions such as in particular in skin care preparations and encompasses e.g. the cosmetic oils.

The exact amount of carrier will depend upon the actual level of the UV-filter substances and any other optional ingredients that one of ordinary skill in the art would classify as distinct from the carrier (e.g., other active ingredients).

In an advantageous embodiment, the cosmetic compositions according to the present invention comprise from 50% to 99%, preferably from 60% to 98%, more preferably from 70% to 98%, such as in particular from 80% to 95% of a carrier, based on the total weight of the cosmetic composition.

In a particular advantageous embodiment, the carrier consists furthermore of at least 30 wt.-%, more preferably of at least 40 wt.-%, most preferably of at least 45 wt.-% of water, such as in particular of 50 to 90 wt.-% of water. Further suitable ranges are from 50 to 75 wt.-%, from 50 to 70 wt.-%, and from 55 to 65 wt.-%.

In particular, the cosmetic compositions according to the present invention are cosmetic or pharmaceutical compositions, preferably cosmetic (non-therapeutic) compositions.

In one embodiment, the cosmetic compositions according to the present invention are applied to mammalian keratinous tissue such as in particular to human skin or the human scalp and hair.

The term “cosmetic composition” as used in the present application refers to cosmetic compositions as defined under the heading “Kosmetika” in Römpp Lexikon Chemie, 10^{th} edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

Preferred cosmetic compositions according to the invention are skin care preparations, decorative preparations, and functional preparations.

Examples of skin care preparations are, in particular, light protective preparations, anti-ageing preparations, preparations for the treatment of photo-ageing, body lotions, body gels, treatment creams, skin protection ointments, balms, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, mascaras or dry and moist make-up formulations.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment, the cosmetic compositions according to the invention are light-protective preparations (sun care products, sunscreens), such as sun protection milks, sun protection lotions, sun protection creams, sun blocks or topical's or day care creams with or without a SPF (sun protection factor) label. Of particular interest are sun protection creams, sun protection lotions and sun protection milks.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing agents, as well as agents to improve elasticity and skin barrier and carriers and/or excipients or diluents conventionally used in cosmetic compositions.

If nothing else is stated, the excipients, additives, diluents, etc. mentioned in the following are suitable for cosmetic compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

In another advantageous aspect, the cosmetic compositions according to the present invention are free of any parabenes, benzethoniumchlorid, piroctone olamine, lauroylarginat, methylisothiazolinon, chlormethylisothiazolinon, bronopol, benzalkoniumchloride, formaldehyd releasing compounds, salicylic acid, triclosan, DMDM hydantoin, chlorphenesin and IPBC (lodopropinylbutyl carbamate).

Preferably, in all embodiments of the present invention, the compositions are free of octocrylene (CAS Number: 6197-30-4).

The term ‘free of octocrylene’ in all embodiments of the present invention means, that no octocrylene is added to the compositions deliberately, i.e. the compound is not detectable with standard analytical means such as HPLC.

In another advantageous aspect, the cosmetic compositions according to the present invention are furthermore free of oxybenzone and/or methylbenzylidenechamphor.

The cosmetic compositions according to the invention may further contain one or more emollients which soothe and soften the skin. As an example, the emollient may be silicone (dimethicone, cyclomethicone), vegetable oils (grape seed, sesame seed, jojoba, etc.), butters (cocoa butter, shea butter), and petrolatum derivatives (petroleum jelly, mineral oil).

In another aspect, the cosmetic composition of the invention may comprise one or more fragrances selected from limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, cinnamal, amyl salicylate, alpha-amylcinnamaldehyde, alpha-methylionone, butylphenylmethylpropional, cinnamal, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, citrus oil, coumarin, diethyl succinate, ethyllinalool, eugenol, Evernia furfuracea extract, Evernia prunastri extract, farnesol, guaiacwood oil, hexylcinnamal, hexyl salicylate, hydroxycitronellal, lavender oil, lemon oil, linalyl acetate, mandarin oil, menthyl PCA, methyl hexadecan, nutmeg oil, rosemary oil, sweet orange oil, terpineol, tonkabean oil, triethyl citrate, vanillin.

The composition of the invention may advantageously comprise moisturizers. Moisturizers are compounds or mixtures of compounds which give cosmetic compositions the quality, after application to or distribution on the skin surface, of reducing the loss of moisture of the stratum corneum (Experimental determination by e.g transepidermal water loss (TEWL)) and/or of positively influencing the hydration of the stratum corneum and/or keeping an actual hydration state.

Non-limiting examples of advantageous moisturizers for use in the present invention include glycerol, lactic acid and/or lactates, especially sodium lactate, biosaccharide gum-1, glycine soya, ethylhexyloxyglycerol, pyrrolidonecarboxylic acid, and urea. Of further advantage, in particular, is the use of polymeric moisturizers from the group of the polysaccharides which are water-soluble and/or swellable in water and/or gellable with the aid of water. Especially advantageous, for example, are hyaluronic acid, chitosan and/or a fucose-rich polysaccharide which is registered in Chemical Abstracts under the registry number 178463-23-5 and is available, for example, under the Fucogel®1000 name from the company SOLABIA S.A. Moisturizers may also be used advantageously as active antiwrinkle ingredients for protection from changes to the skin of the kind occurring in skin aging, for example.

The cosmetic compositions of the invention may further comprise advantageously, although not mandatorily, fillers which have the effect, for example, of further improving the sensorial and cosmetic properties of the formulations and evoking or intensifying a velvety or silky skin sensation, for example. Advantageous fillers in the sense of the present invention are starch and starch derivatives (such as tapioca starch, distarch phosphate, aluminum or sodium starch octenylsuccinate, and the like, for example), Valvance pigments which have neither primarily UV filter effect nor coloring effect (such as Valvance Touch 210 or 250 for example) and/or Aerosils® and/or talc and/or polyethylene, nylon, and silica dimethyl silylate.

The water phase of the compositions of the invention may advantageously comprise customary cosmetic auxiliaries, such as, for example, alcohols, particularly those of low C number, preferably ethanol and/or isopropanol, or polyols of low C number, and also ethers thereof, preferably glycerol, electrolytes, self-tanning agents, and also, in particular, one or more thickeners, which may be advantageously selected from the group of silicon dioxide, aluminum silicates, polysaccharides and/or derivatives thereof, e.g., hyaluronic acid, xanthan gum, hydroxypropylmethylcellulose as well as particularly advantageously from the group of polyacrylates, preferably a polyacrylate from the group referred to as carbopols, examples being carbopols of types 980, 981, 1382, 2984, and 5984, in each case individually or in combination. Further thickeners advantageous in accordance with the invention are those having the INCI designation Acrylates/C10-30 Alkyl Acrylate Crosspolymer (e.g., Pemulen TR 1, Pemulen TR 2, Carbopol 1328 from NOVEON) and also Aristoflex AVC (INCI: Ammonium Acryloyldimethyltaurate/VP Copolymer) as well as Simugel NS (INCI: Hydroxyethyl Acrylate/Sodium Acryloyldimethyl Taurate Copolymer & Squalane & Polysorbate 60). Further suitable thickeners in all embodiments of the present invention are biopolymers like microcrystalline cellulose, cellulose, cellulose gum, sphingomonas ferment extract.

It is preferred in accordance with the invention if the composition comprises one or more thickeners aselected from the group consiting of xanthan gum, microcrystalline cellulose, cellulose, cellulose gum, crosslinked acrylate/C10-C30 alkyl acrylate polymer, hydroxyethyl acrylate/sodium acryloyldimethyl taurate copolymer and/or vinylpyrrolidone/hexadecane copolymer, preferably xanthan gum and/or hydroxyethyl acrylate/sodium acryloyldimethyl taurate, most preferably both of xanthan gum and hydroxyethyl acrylate/sodium acryloyldimethyl taurate.

The (total) amount of the one or more thickener in the compositions according to the present invention is preferably selected in the range from 0.1 to 1 wt.-%, preferably from 0.1 to 1.5 wt.-%, most preferably from 0.1 to 0.1 wt.-%, such as in the range of 0.25 to 1 wt.-%, based on the total weight of the composition.

In another aspect, the composition may have an SPF of at least 15, preferably at least 20, most preferably of at least 30.

Advantageously in accordance with the invention, the composition of the invention may comprise further film formers.

It is especially advantageous to select said further film formers from the group of the polymers based on polyvinylpyrrolidone (PVP), as well as Capryloyl Glycerin/Sebacic Acid Copolymer, Diisostearoyl Polyglyceryl-3 Dimer Dilinoleate and/or Ethyl Cellulose.

Particular preference is given to copolymers of vinylpyrrolidone, as for example the PVP hexadecane copolymer and the PVP eicosene copolymer, which are available under the trade names Antaron V216 and Antaron V220 from GAF Chemicals Corporation.

Likewise advantageous as further polymeric film formers are for example, sodium polystyrene sulfonate, which is available under the trade name Flexan 130 from National Starch and Chemical Corp., and/or polyisobutene, available from Rewo under the trade name Rewopal PIB1000. Examples of further suitable polymers are polyacrylamides (Seppigel 305), polyvinyl alcohols, PVP, PVP/VA copolymers, polyglycols, acrylate/octylacrylamide copolymer (Dermacryl 79) Likewise advantageous is the use of hydrogenated castor oil dimer dilinoleate (INCI Hydrogenated Castor Oil Dimer Dilinoleate), which can be acquired from Kokyu Alcohol Kogyo under the name Risocast DA-H, or else PPG-3 benzyl ether myristate, which can be acquired under trade name Crodamol STS from Croda Chemicals.

The cosmetic compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The amount of the topical sunscreen emulsion to be applied to the skin is not critical and can easily be adjusted by a person skilled in the art. Preferably the amount is selected in the range of 0.1 to 3 mg/cm2 skin, such as preferably in the range of 0.1 to 2 mg/cm2 skin and most preferably in the range of 0.5 to 2 mg/cm2 skin.

In accordance with the invention is the use of the sunscreens of the invention for protection from skin aging (especially for protection from UV-induced skin aging) and also as a sun protection composition.

Finally, a subject-matter of the invention is a method for the cosmetic treatment of keratinous substances such as in particular the skin, wherein a composition as defined herein is applied to the said keratinous substances such as in particular to the skin. IF UV-filter substances are present in said compositions, said method is in particular suitable to protect the skin against the adverse effects of UV-radiation such as in particular sun-burn and/or photoageing while avoiding florescence stains on garment and/or clothing.

The following examples are provided to further illustrate the compositions and effects of the present invention. These examples are illustrative only and are not intended to limit the scope of the invention in any way.

### Experimental Part

### Formulations

The formulations (O/W emulsions) as outlined in table 1 have been prepared according to standard methods in the art.

### (A) Water Repellency Test

1) apply 40 mg of the formulation in small droplets on Schönberg 2 mü plates (5 cm×5 cm) and distribute the formulation homogenously
2) store the plates at 40° C. for 15 minutes
3) place a 50 microliter water droplet on the plate, wait for 1 minute and take a picture
4) measure the diameter of the droplet
5) for each formulation 3 droplets are measured.

TABLE 1
Cosmetic O/W emulsions

| | | | Reference | | Invention | | |
|---|---|---|---|---|---|---|---|
| # | Tradename | INCI | 1 | 2 | 1 | 2 | 3 |
| A | Water Dem | aqua; | Ad 100 | Ad 100 | Ad 100 | Ad 100 | Ad 100 |
| A | Keltrol CG-T | xanthan gum; | 0.25 | 0.25 | 0.25 | 0.25 | 0.25 |
| B | AMPHISOL ® K | potassium cetyl | 2.50 | 2.50 | 2.50 | 2.50 | 2.50 |
| | | phosphate; | | | | | |
| B | Lanette 18 | stearyl alcohol; | 2.50 | 2.50 | 2.50 | 2.50 | 2.50 |
| B | Lanette O | cetearyl alcohol; | 1.00 | 1.00 | 1.00 | 1.00 | 1.00 |
| B | Finsolv TN | c12-15 alkyl | 15.00 | 28.00 | 15.00 | 15.00 | 15.00 |
| | | benzoate; | | | | | |
| B | Oxismooth CP | isoamyl | 13.00 | — | 13.00 | 13.00 | 13.00 |
| | | caprylate/caprate; | | | | | |
| B | Sepinov EMT 10 | hydroxyethyl | 0.50 | 0.50 | 0.50 | 0.50 | 0.50 |
| | | acrylate/sodium | | | | | |
| | | acryloyldimethyl | | | | | |
| | | taurate copolymer; | | | | | |
| C | Euxyl PE 9010 | phenoxyethanol & | 1.00 | 1.00 | 1.00 | 1.00 | 1.00 |
| | | ethylhexylglycerin | | | | | |
| D | Tilamar PDO | propanediol; | — | — | 3.00 | — | — |
| D | Hydrolite 5 | pentylene glycol; | — | — | — | 3.00 | — |
| | Green | | | | | | |
| D | 1,3 Butylene | butylene glycol; | — | 3.00 | — | — | 3.00 |
| | Glycol cos | | | | | | |
| | grade | | | | | | |

| | | | | | |
|---|---|---|---|---|---|
| Average droplet diameter [cm] | 1.21 | 1.21 | 1.13 | 1.07 | 1.07 |
| Improvement of hydrophobicity vs. Ref | — | — | +6.6% | +11.6% | +11.6% |

As can be retrieved from the results in table 1, compositions according to the present invention exhibit an improved water repellency compared to the reference formulation.

TABLE 2
Sunscreen composition

| | | | Invention | | |
|---|---|---|---|---|---|
| # | Tradename | INCI | 4 | 5 | 6 |
| A | Water Dem | aqua | Ad 100 | Ad 100 | Ad 100 |
| A | Keltrol CG-T | xanthan gum | 0.25 | 0.25 | 0.25 |
| A | 1,3 Butylene Glycol | butylene glycol | 3.00 | 3.00 | 3.00 |
| | cos grade | | | | |
| B | AMPHISOL ® K | potassium cetyl phosphate | 2.50 | 2.50 | 2.50 |
| B | Lanette 18 | stearyl alcohol | 2.50 | 2.50 | 2.50 |
| B | Lanette O | cetearyl alcohol | 1.00 | 1.00 | 1.00 |
| B | Finsolv TN | C12-15 alkyl benzoate | 15.00 | 15.00 | 15.00 |
| B | Oxismooth CP | isoamyl caprylate caprate | 13.00 | 13.00 | 13.00 |
| B | PARSOL ® Shield | bis-ethylhexyloxyphenol | 2.50 | 2.5 | 2.5 |
| | | methoxyphenyl triazine | | | |
| B | PARSOL ® 1789 | butyl methoxydibenzoylmethane | — | 4.5 | — |
| B | DHHB | diethylamino hydroxybenzoyl hexyl | — | — | 5.0 |
| | | benzoate | | | |
| B | Sepinov EMT 10 | hydroxyethyl acrylate/sodium | 0.50 | 4.50 | 4.50 |
| | | acryloyldimethyl taurate copolymer | | | |
| C | Euxyl PE 9010 | phenoxyethanol & | 1.00 | 1.00 | 1.00 |
| | | ethylhexylglycerin | | | |

| | | | |
|---|---|---|---|
| Average droplet diameter [cm] | 1.03 | 0.89 | 0.90 |
| Improvement of hydrophobicity vs. Reference | +14.9% | +26.5% | +25.6% |

As can be retrieved from the results in table 2, the addition of BEMT, BMDBM and DHHB further ameliorates the water repellency.

### (B) Sensory Evaluation

All sensory evaluations have been perfomed using the emulsions shown in table 1 and 2. The determination of the sensorial features was then conducted according to a descriptive sensory evaluation process for the parameters outlined in table 3 by well-trained panelists. 50 μl of the respective emulsion of table 1 and 2 were applied on a spot on the forearm surface. After application the respective emulsion was rubbed in until the product was absorbed by skin. The parameters were evaluated immediately after the rub in phase. The results are depicted in table 3 below.

TABLE 3
Result of the sensory evaluation (sensorial attractivity)

| | Reference | | Invention | | | | | |
|---|---|---|---|---|---|---|---|---|
| Sensorial Attribute | 1 | 2 | 1 | 2 | 3 | 4 | 5 | 6 |
| Stickiness | + | ± | + | + | ++ | ++ | ++ | + |
| Silkiness | ± | + | + | ++ | + | + | ++ | + |
| Skin whitening during spreading | + | + | ++ | ++ | + | ++ | + | ++ |
| Overall sensorial attractivity | + | + | ++ | ++ | ++ | ++ | ++ | ++ |

± OK
+ good
++ very good

As can be retrieved from the results in table 3, the compositions according to the present invention exhibit a superior skin feel and/or look compared to the references resulting in an overall improved sensorial attractivity.

1. A cosmetic composition comprising an alkandiol and one or more cosmetic oils, wherein at least one of the cosmetic oils is isoamyl caprylate caprate.

2. The cosmetic composition according to claim 1, wherein the amount of alkanediol(s) is selected in the range from 1 to 10 wt.-%, preferably from 1.5 to 7.5 wt.-%, most preferably from 2 to 6 wt.-%, such as in the range of 2 to 5 wt.-%, based on the total weight of the composition

3. The cosmetic composition according to claim 1, wherein the amount of isoamyl caprylate caprate in the composition is selected in the range from 5 to 30 wt.-%, more preferably from 7.5 to 20 wt.-%, most preferably from 10 to 15 wt.-%, based on the total weight of the composition.

4. The cosmetic composition according to claim 1, wherein the weight-ratio of isoamyl caprylate caprate to alkandiol(s) is selected in the range from 2.5:1 to 20:1, preferably from 4:1 to 10:1.

5. The cosmetic composition according to claim 1, wherein at least one further cosmetic oil selected from the group consisting of C₁₂-C₁₅ alkyl benzoate, dicaprylate/dicaprate, phenethyl benzoate, dibutyl adipate, diisopropyl sebacates, dicaprylyl carbonate, di-C₁₂₋₁₃ alkyl tartrates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol and ethylhexyl cocoates is present.

6. The cosmetic composition according to claim 1, wherein the at least one further cosmetic oil is C₁₂-C₁₅ alkylbenzoate.

7. The cosmetic composition according to claim 5, wherein the weight-ratio of the at least one further cosmetic oil to isoamyl caprate caprylate is selected in the range from 5:1 to 1:5, preferably from 2.5:1 to 1:2.5, most preferably from 1.5:1 to 1:1.5.

8. The cosmetic compositions according to claim 1, wherein the composition is an O/W emulsion comprising an oily phase dispersed in an aqueous phase in the presence of an O/W emulsifier, preferably in the presence of a cetyl phosphate emulsifier, most preferably in the presence of potassium cetyl phosphate.

9. The cosmetic composition according to claim 1, wherein the cosmetic composition further comprises bis-ethylhexyloxyphenol methoxyphenyl triazine.

10. The cosmetic composition according to claim 9, wherein the amount of bis-ethylhexyloxyphenol methoxyphenyl triazine in the composition is selected in the range from 0.1 to 10 wt.-%, preferably from 0.5 to 5 wt.-%, most preferably from 1 to 5 wt.-%, based on the total weight of the composition.

11. The cosmetic composition according to claim 1, wherein the composition further comprises butylmethoxy dibenzoylmethane and/or diethylamino hydroxybenzoyl hexyl benzoate.

12. The cosmetic composition according to claim 1, wherein the composition further comprises one or more a preservative/preservative booster selected from the group consisting of phenoxyethanol and/or ethylhexylglycerin.

13. The cosmetic composition according to claim 1, wherein the composition further comprises one or more of behenyl alcohol, cetyl alcohol, cetearyl alcohol, stearyl alcohol and glyceryl stearate, preferably of behenyl alcohol, stearyl alcohol and cetearyl alcohol, most preferably cetearyl alcohol and stearyl alcohol.

14. Use of an alkandiol for increasing the water repellency of a cosmetic composition comprising isoamyl caprylate caprate.

15. A method of improving the water repellency of a cosmetic compositions, said method encompassing the step of preparing a cosmetic composition comprising both of an alkandiol and isoamyl caprylate caprate, applying said composition to the skin, allowing the volatile constituents to evaporate thereby forming a hydrophobic film and optionally appreciating the effect.
